(19) United States
(12) Patent Application Publication
(10) Pub. No.: US 2017/0182287 A1
(43) Pub. Date: Jun. 29, 2017

(54) INTRODUCER FOR ACCESSING CORONARY SINUS VIA RIGHT PARASTERNAL MEDIASTINOTOMY

(71) Applicant: THE TRUSTEES OF COLUMBIA UNIVERSITY IN THE CITY OF NEW YORK, New York, NY (US)

(72) Inventor: Henry M. Spotnitz, New York, NY (US)

(21) Appl. No.: 15/397,379

(22) Filed: Jan. 3, 2017

Related U.S. Application Data
(63) Continuation of application No. PCT/US15/39064, filed on Jul. 2, 2015.
(60) Provisional application No. 62/020,907, filed on Jul. 3, 2014.

(51) Int. Cl. A61M 25/00; A61M 25/01; A61B 5/042; A61N 1/05

(52) U.S. Cl. CPC A61M 25/0041; A61N 1/056; A61M 25/0102; A61B 5/042; A61M 25/0026; A61N 2001/0585; A61M 2025/0004; A61M 2210/125; A61M 2025/0036

(57) ABSTRACT

An introducer configured with a first curve having a first angle that traverses space of an atrial appendage, a central atrium, caudad to the coronary sinus, and a second curve that has an angle sufficient to align the introducer with an intrinsic curvature of the coronary sinus of a subject.

### CROSS-REFERENCE TO RELATED APPLICATIONS

This application is a continuation of International Application No. PCT/US15/39064, filed Jul. 2, 2015, which claims the benefit of U.S. Provisional Application No. 62/020,907, filed Jul. 3, 2014, the contents of each are hereby incorporated by reference in their entirety.

### TECHNICAL FIELD

The disclosed subject matter describes an introducer configured to access the coronary sinus via a right parasternal mediastinotomy and deliver therapeutic agent or a medical device to the coronary sinus.

BACKGROUND

CRT reduces morbidity and mortality in congestive heart failure and can improve cardiac function and cardiac output without increasing myocardial oxygen consumption. Challenges to CS lead insertion include locating the CS, lead delivery and stability, vein selection, and avoidance of diaphragmatic pacing. Technical failure of CRT implementation is reported in 8% to 13% of attempts. Variation in cardiac venous anatomy contributes obstacles. The endocardial approach to the CS is limited by difficulty directing flexible catheters over relatively long distances from the left subclavian vein. Steerable catheters are available, but angulation and leverage are often insufficient to reach posterior CS locations.

CS lead insertion is technically difficult in many patients. Fatemi et al averaged 43±29 minutes to position an LV lead in the CS. Implant time exceeded 72 minutes in 16% of patients. Alternate strategies might improve mechanical advantage, approach angles, and spatial mobility. Too much leverage with standard endocardial cannulas introduces a risk of prolapse, buckling of the guiding catheter, and lead extrusion.

Over 100,000 annual CS lead insertions for CRT in the United States implies 8,000-13,000 failures of endocardial insertion. Improved CS access could provide better care and reduce the cost of health care delivery for these patients while providing scientific benefits through better understanding of CS localization and lead position. LV lead location is considered important in CRT optimization. In dilated cardiomyopathy, the optimum location is the latest activated and contracting LV segment. However, in ischemic cardiomyopathy, LV lead placement should avoid regions of slow conduction and associated injury.

Faster and more reliable CS access is needed and could lead to important diagnostic and therapeutic advances. In particular, there is a need for a device to permit CS access from the right parasternal mediastinotomy, which can provide advantages including: (1) Physical proximity to the coronary sinus (CS); (2) more flexibility in approach angles, leverage, imaging, (3) less size limitation vs. endocardial—allows echocardiography probes and multichannel prototypes; (4) applicability to all CS interventions, including stents, mechanical circulatory assistance, CS occlusion; and (5) adaptability for totally percutaneous intervention.

The present disclosure satisfied those needs.

### SUMMARY

In one aspect of the present disclosure, an introducer is configured to access a coronary sinus from an incision proximate to a right atrium appendage. The introducer includes a tubular member including a first curve in a first plane corresponding to a curve of a left ventricle portion of the coronary sinus and a second curve in a different second plane corresponding to a curve of a branch vein of the coronary sinus. The first curve is disposed along a length of the tubular member and has a first angle that traverses space of an atrial appendage, a central atrium, caudad to the coronary sinus. The second curve has an angle sufficient to align the introducer with an intrinsic curvature of the coronary sinus of a subject.

Implementations of the disclosure may include one or more of the following features. The first angle may include an angle of about 40 to 50 degrees, and the second angle may include an angle of about 15 to 25 degrees. The second angle may align the introducer with the intrinsic curvature at the origin of the coronary sinus. The introducer may be configured to access the coronary sinus via right parasternal mediastinotomy. In some implementations, the tubular member may include an outer lumen and a central lumen. In some implementations, the tubular member may include a central lumen having a diameter of about 9 French, coaxially disposed within an outer lumen having a diameter of about 20 French. In other implementations, the tubular member may include a central lumen having a diameter of about 2 French, coaxially disposed within an outer lumen having a diameter of about 13 French.

In some implementations, an additional lumen may be disposed between the outer lumen and the central lumen. A first lumen may be configured to deliver a therapeutic agent to the coronary sinus, and a second lumen is configured to deliver a medical device to the coronary sinus. The medical device may be selected from a group consisting of: a ring, a lead, and a stent or a combination thereof. In other implementations, one lumen can be configured to introduce a stylet to the coronary sinus and another lumen can be configured to introduce a lead to the coronary sinus. In still other implementations, the tubular member may include a steerable distal tip, in which the steerable tip is formed of a material having a different durometer than the tubular member. The tubular member can be formed from SMART™ material, and the first and second curves can be formed in situ.

In yet another aspect of the disclosure, an introducer is configured to access a coronary sinus from an incision proximate to a right atrium appendage. The introducer includes a tubular member including a first curve in a first plane corresponding to a curve of a left ventricle portion of the coronary sinus and a second curve in a different second plane corresponding to a curve of a branch vein of the coronary sinus, in which the tubular member includes a central lumen having a diameter of about 9 French, coaxially disposed within an outer lumen having a diameter of about 20 French.

In another aspect of the disclosure, an introducer is configured to access a coronary sinus from an incision proximate to a right atrium appendage. The introducer includes an outer tubular member including a first curve in a first plane corresponding to a curve of a left ventricle portion of the coronary sinus and a second curve in a different second plane corresponding to a curve of a branch vein of the coronary sinus, and an inner tubular member coaxially disposed within the outer tubular member, in which at least one lumen is disposed between the inner and outer tubular members.

In still another aspect of the disclosure, a method for accessing the coronary sinus from a right parasternal mediastinotomy includes forming a parasternal medial sternotomy on a subject; inserting an introducer comprising an inner tubular member and an outer tubular member into the sternotomy; in which at least the outer tubular member includes a first curve along its length disposed in a first plane that traverses space from an atrial appendage to a central atrium to the coronary sinus, and a second curve disposed in a different second plane sufficient to align the introducer with an intrinsic curvature of the coronary sinus; accessing the coronary sinus; and inserting a therapeutic agent or a medical device through a lumen of the introducer during surgery.

In some implementations, the method may include delivering a therapeutic agent to the coronary sinus. In other implementations, the method may include delivering a medical device to the coronary sinus. The medical device may be selected from a group consisting of: a ring, a lead, and a stent or a combination thereof.

The details of one or more implementations are set forth in the accompanying drawings and the description below. Other features, objects, and advantages will be apparent from the description and drawings, and from the claims.

### BRIEF DESCRIPTION OF THE DRAWINGS

A detailed description of various aspects, features and embodiments of the subject matter described herein is provide with reference to the accompanying drawings, which are briefly described below. The drawings are illustrative and are not necessarily drawn to scale, with some components being exaggerated for clarity. The drawings illustrate various aspects and features of the present subject matter and may illustrate one or more embodiment(s) or example(s) of the present subject matter in whole or in part. Together with the description, the drawings serve to explain the principles of the disclosed subject matter.

FIG. 1 is a schematic of an introducer in accordance with the disclosed subject matter.

FIG. 2 is a schematic of a cross section of an embodiment of the introducer in accordance with the disclosed subject matter

FIG. 3 is a schematic of a cross section of a second embodiment of the introducer in accordance with the disclosed subject matter.

FIG. 4 is an illustration of an introducer comprising a bend angle, hook angle and twist angle in accordance with an embodiment of the disclosed subject matter.

FIG. 5 is an illustration of the introducer of FIG. 4 introduced through the vascularture of a heart.

FIG. 6 is an illustration of another introducer comprising a bend angle, hook angle and twist angle in accordance with an embodiment of the disclosed subject matter.

FIG. 7 is a table indicating various parameters for the design of an introducer in accordance with the disclosed subject matter.

It will be apparent to those skilled in the art that various modifications and variations can be made to the apparatus and methods of the disclosed subject matter without departing from the spirit or scope of the disclosed subject matter. Such modifications and variations are to be construed as within the scope of the appended claims.

### DETAILED DESCRIPTION OF EXEMPLARY EMBODIMENTS

In accordance with an aspect of the subject matter described, parasternal mediastinotomy access to the coronary sinus of a subject for cardiac resynchronization, mitral annuloplasty, and delivery of therapeutics, medical devices, or instrumentation is described. The technology can facilitate a number of cardiovascular procedures.

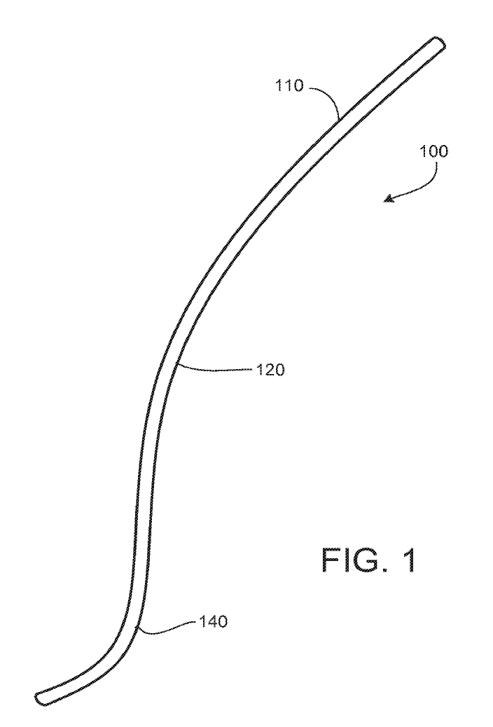

In accordance with an aspect of the described subject matter, as shown in FIG. 1 an introducer 100 configured to access a coronary sinus from an incision proximate to a right atrium appendage is provided, such as for example, by a right parasternal mediastinotomy. The introducer comprises a tubular member 110 including a first curve 120 in a first plane corresponding to a curve of a left ventricle portion of the coronary sinus and a second curve 130 in a different second plane corresponding to a curve of a branch vein of the coronary sinus. In one embodiment, the first curve is disposed along a length of the tubular member and has a first angle that traverses space of an atrial appendage, a central atrium, caudad to the coronary sinus. The second curve has an angle sufficient to align the introducer with an intrinsic curvature of the coronary sinus of a subject.

In one embodiment, the first angle includes an angle of about 40 to 50 degrees, and the second angle includes an angle of about 15 to 25 degrees. However, other angles may be employed. For example, other angles that allow the second angle to align the introducer with the intrinsic curvature at the origin of the coronary sinus can be used. In this regard, the introducer can be personalized to a particular subject. The intrinsic curvature can be visualized by imaging methods available to one of ordinary skill in the art to discern the second angle.

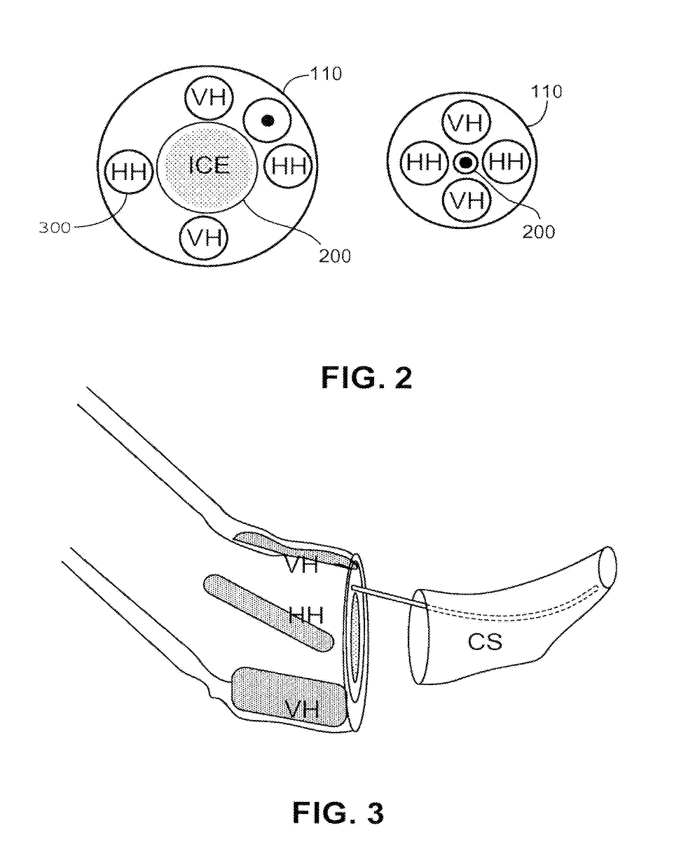

As shown in FIGS. 2, the introducer comprises an inner tubular member 200 coaxially disposed within an outer tubular member 110. Accordingly, the introducer has at least a central lumen formed along the length of the inner tubular member. In some embodiments, additional lumen 300 are defined between the inner and outer tubular members. In this regard, the lumen defined along the length of the introducer provide a passageway to the coronary sinus of a subject. For example, a first lumen can be configured to deliver therapeutic agent directly to the coronary sinus. A second lumen can be configured to deliver a medical device to the coronary sinus, such as a lead, stent, ring, or other medical device. The introducer can also be used for coronary sinus blood sampling. For example, the lumen can be used to introduce an aspirator for aspirating the coronary sinus blood. This application may be helpful for insight into local cardiac pathophysiology and treatment.

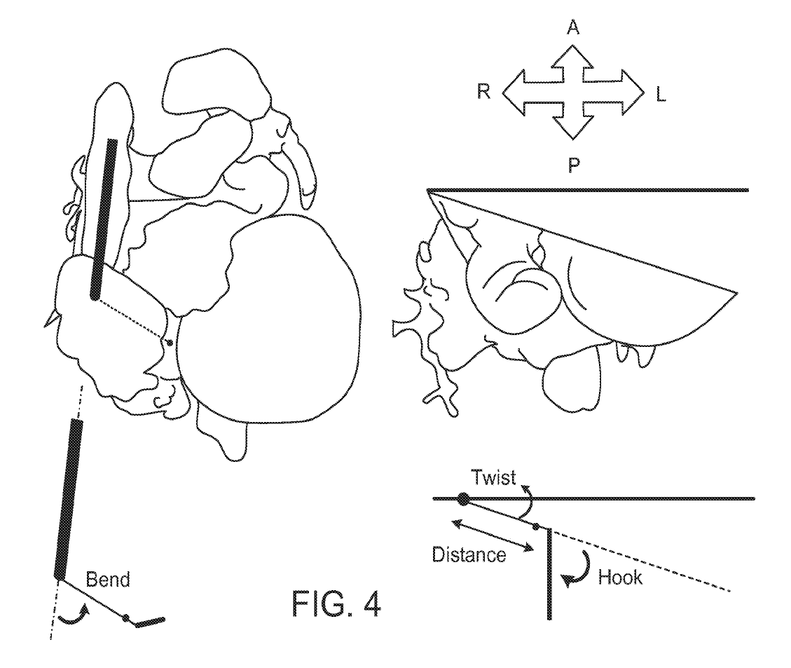

As illustrated in FIG. 3, an imaging probe can be used to visualize the introducer facilitate alignment of the introducer with an orifice of the coronary sinus. One or more inflatable members may be employed to align the path of the introducer into the contrary sinus.

In one embodiment, the introducer body includes an outer tubular member, an inner tubular member and secondary tubular members disposed between inner and outer tubular members. Each tubular member defines a lumen along the length of the introducer body. For example, an introducer may include an outer tubular having an outer diameter of 20 French (F), and inner coaxially disposed tubular member having an outer diameter of 9 F. A plurality of secondary tubular members may be disposed between the inner and outer tubular members. The secondary tubular members may have outer diameters between 13 and 19 F, preferably 9 F. In another embodiment, the outer tubular body.

In another aspect, a steerable introducer is provided. In this aspect, the introducer body is formed from SMART™ material, such that the first and second curves described above are formed in situ. SMART materials may include but are not limited to nitinol,

In yet another aspect, a method for accessing the coronary sinus from a right parasternal mediastinotomy is provided. The method comprises forming a parasternal medial sternotomy on a subject, inserting an introducer comprising an inner tubular member and an outer tubular member into the sternotomy, wherein at least the outer tubular member includes a first curve along its length disposed in a first plane that traverses space from an atrial appendage to a central atrium to the coronary sinus, and a second curve disposed in a different second plane sufficient to align the introducer with an intrinsic curvature of the coronary sinus. The introducer provides access to the coronary sinus, such that delivery of a therapeutic agent or a medical device may be advanced into the coronary sinus through a lumen of the introducer during surgery.

For example but not limitation, the medical device may be a stent or reduction stent, a lead, a guidewire, or an aspirator for aspirating coronary sinus blood.

One advantages of parasternal mediastinotomy includes sufficient space for insertion of large introducers. Moreover, described herein is a “personalized introducer,” with curvatures, dimensions, and refinements customized for the individual patient.

Specific Aim 1. Introducer Development and Testing. This includes geometry optimization in 66 heart failure patients, with the aid of 1.5 Tesla magnetic resonance imaging (MRI) and x-ray computerized tomography (CT). Digitized images will be processed with 3-D modeling software to define curvatures and segment lengths needed to pass introducers from the appendage of the right atrium to the CS. Results will determine the most accurate and cost effective imaging for fixed or customized introducers. Additionally, in silica testing will determine which fixed introducers are most widely applicable, and testing will seek to bring the costs of customized introducers within an economically viable range.

Specific Aim 2: Validation in Pigs. Prototype introducers will be tested and further developed in six domestic pigs and phantoms. This testing will examine integrated imaging, introducer steering, and CS cannulation. Technologies investigated will include commercial steerable catheters and real time imaging incorporating commercial intracardiac echocardiography (ICE).

The CS provides unique anatomic and physiologic proximity to all four cardiac chambers and the coronary circulation. CS cannulation is used for infusion of myocardial protectants during cardiac surgery. Access is facilitated by bimanual manipulation of the heart and cannula or by direct visualization of the CS orifice. This retrograde infusion assures global cooling and minimizes myocardial injury related to obstruction of flow by coronary artery disease. CS access is a/so utilized for clinical trials of infarct reduction by pressure controlled intermittent CS occlusion and for closed chest mitral annuloplasty using nitinol CS rings. In electrophysiology, CS access is employed for diagnostic studies and for atrial and ventricular arrhythmia ablation. Hundreds of thousands of electrophysiology procedures are performed annually. In addition, an estimated 100,000 left ventricular pacing leads are inserted annually into the CS for biventricular pacing in cardiac resynchronization therapy (CRT). CRT improves the synchrony of contraction of the free wall of the left ventricle in heart failure and requires right atrial and right and left ventricular (L V) leads.

In cardiac pacing, venous obstruction and congenital heart disease may require innovative approaches to cardiac access. Parasternal mediastinotomy is an innovative, minimal access approach to the right atrium. We have utilized this approach clinically for RV lead insertion and find it effective and well tolerated, with little postoperative pain, despite resection of an anterior rib cartilage. Because the patient is supine during the procedure, hemodynamic embarrassment associated with lateral thoracotomy is avoided. Chamberlain originally developed this incision for biopsy of anterior mediastinal tumors. This approach was used for epicardial pacing lead insertion in 1976 and has been useful under local anesthesia if supported by positive pressure ventilation. Parasternal mediastinotomy has been used for coronary artery bypass surgery in pigs. We described LV lead insertion through a CS cardioplegia cannula in pigs and used echocardiography clinically to position a lead in the CS after fluoroscopic guidance failed.

Current approaches to transvenous lead insertion via the CS employ three or more preformed configurations of 9 trench polyurethane or teflon cannulas with deformable obturators. A small lumen passes a flexible guidewire several centimeters into the coronary sinus. The obturator and cannula are advanced over the guidewire to a stable location in the CS and are then removed and replaced with a balloon catheter to occlude the CS for retrograde angiography. The angiogram is used to select target branches for lead insertion, using an over-the wire technique. The leads are quite delicate, 6 trench or less, and may contain multiple pacing electrodes. The lead tip must be wedged securely into the target vein. The cannula stabilizes the advancing portion of the lead. Excessive force can cause the cannula to buckle and prolapse out of the CS, so that the whole procedure must be repeated. CS lead insertion requires considerable skill. Complications are uncommon but include cardiac perforation, CS trauma and dissection, lead displacement, inadequate pacing thresholds and/or sensing, and phrenic nerve pacing. A steerable catheter can help locate the CS, for example, the Marinr (Multi-Curve) and MCXL Series (Medtronic, Inc., Minneapolis, Minn.).

Steerable catheters, used for precise, stable localization helps with arrhythmia ablation, coronary and neurovascular stenting, and occlusion of arteriovenous fistulas or small arteries feeding internal hemorrhage. Steerable catheters ameliorate issues with friction and angulated vascular paths that can render passage of catheters traumatic and unpredictable. A variety of materials and techniques are available, including hydraulics, magnetic fields, internal pullwires and alloys (e.g. nitinol), ceramics and plastics that change configuration with temperature or electrical stimulation. The most highly developed are the Sensei and Stereotaxis (niobe) systems which are remotely controlled via consoles.

The initial target for the proposed device is patients requiring epicardial lead insertion by thoracotomy after CS lead insertion fails. Such patients usually undergo robotic or minimal access or full thoracotomy for cardiac exposure. This is straightforward in a primary operation but can be quite challenging after one or multiple previous thoracotomies. Dense intrapericardial adhesions then may obscure anatomical landmarks, increasing risk of cardiovascular injury and/ or bleeding when adhesions are divided. Epicardial lead insertion in this setting is daunting, and multiple site testing may be needed to secure adequate electrical properties. This surgery is tedious, carries risk of infection, shock and arrhythmias. Long-term lead utility is unpredictable.

The spectrum of patients referred for lead insertion by thoracotomy ranges from a long surgical history and extensive pleuropericardial adhesions to no adhesions and no previous surgery or myocardial infarction. For the simplest group, minithoracotomy and robotic approaches are available. However, endocardial leads in general are more reliable for long-term use, in part because of high flexion stresses and ongoing fibrosis at the epicardial surface. Our plan utilizes a minithoracotomy to expose and puncture the right atrium. The CS is located within the right atrium and a standard endocardial CS lead is inserted. Performance of these leads should equal the best current endocardial leads and be superior to epicardial leads. Complications of this surgery include bleeding, death, infection, lead displacement, CS dissection, cardiac perforation, and failure to implant a usable LV lead.

Faster, more reliable CS access would create options for diagnostic and therapeutic advances. Our objective is to access the CS via a parasternal approach using a patient-tailored catheter that will simplify procedures and increase accuracy while decreasing risks of cardiac interventions. On aspect of the present disclosure focuses on changing the clinical practice of pacemaker insertion for heart failure. Parasternal lead insertion is expected to be superior to any current method of epicardial lead insertion in speed, efficacy, physiologic benefit, and limitation of morbidity and discomfort. These advantages will be accentuated if intrapericardial adhesions are present.

The introducer provides an alternative approach to CS cannulation, which can be used for research, diagnosis, and therapy. Parasternal approaches have previously been established for right atrial and ventricular pacemaker lead insertion when conventional venous paths are obstructed. This technique is most likely to be adopted by electrophysiologysurgery teams.

In another aspect, use of 3-D imaging, modeling, and printing can optimize introducers for complex intracardiac geometry. Testing in silico of the fit of previously derived cannula shapes to new MRI and CT images of candidates for CS lead insertion can be implemented. Further, incorporation of intracardiac echocardiography to provide real-time image feedback on CS localization may be used.

### EXAMPLE 1

Patient-specific identifiers are removed and replaced by a study number. Images are archived on DVD-R optical disks and transferred to the company. Three-dimensional modeling performed there uses an encrypted Windows digital computer running Materialise Interactive Medical Image Control System (MIMICS) software (Materialise NV). MIMICS creates a three-dimensional surface map by segmentation of the DICOM image, using a “marching cubes” algorithm. The map is calibrated around a selected voxel representing the average optical density for the entire data set. Results are recorded in STL file format. The STL surface model is next opened in SOLIDWORKS (Dassault Systemes SOLIDWORKS Corp., Waltham Mass.) computer aided design software.

Introducer Configuration. Separate vectors are manually aligned with the long axis of the superior vena cava and the orifice of the CS. The critical path from the cava to the CS is then defined by manual fitting, usually involving vector segments oriented in three principal directions. Junctions of these vector segments are smoothed for compatibility with fabrication, and a final segment from the atrial appendage to the introducer path is added. The data describing the desired catheter shape is then transferred to a three-dimensional printer or pipe bender for fabrication of the final product. For thermoforming, introducers are heated and cooled in the mold, retaining the required shape. The obturator hub is marked to indicate the expected orientation of the CS.

Configuration Testing. Fifty-one digital files were previously created describing the 3-D path from the atrial appendage to the coronary sinus in patients with CS leads previously implanted. These will be reduced to a subset of twenty representative of the range available. These will be tested for fit visually in SOLIDWORKS against each of the new data sets obtained for heart failure patients who have not undergone lead insertion. A scoring system of 1 to 5 will be assigned to the five best fitting configurations. The lowest average score will identify the most useful configuration. Results will be reviewed to determine which configurations are most useful and whether a defined set of preconfigured cannulas is an acceptable alternative to custom designed cannulas. Results of this testing will be reviewed by all investigators.

Aim 2. This Aim will validate introducers in pigs and phantoms. A postmortem exam will detect any surgical complications. The heart is fixed at physiologic filling pressures. Technologies to be evaluated include steerable catheter and integral intracardiac echocardiography (ICE). These will be tested initially in plastic phantoms and fixed hearts, advancing to pig studies if successful. Polyurethane 20 trench extrusions (Zeus, Inc. Orangeburg S.C.) customized to our specifications would be used for testing of in-house customized technologies for catheter steering and intracardiac imaging. in vitro testing of in-house options for vectoring the introducer include shaped firm guide wires, pullwire devices, hydraulic balloon or nitinol arrays. Intracardiac Echocardiography (ICE) is only one possible form of intracardiac imaging that would be feasible in this project, others including intravascular ultrasound catheters and a variety of approaches to visualizing cardiac structures through blood.

Commercial ICE probes are rather rigid and unlikely to match the curvatures of these introducers. Options include a bifurcacated introducer structure with rigid imaging and separate flexible segments. Alternatively, ICE could be introduced separately from below. The Sensei and Stereotaxis systems are far too expensive to be purchased or leased for technology trials. We would hope to gain access to one or both of these through a cooperative agreement.

Surgery will be performed on six pigs by a team experienced with parastermal mediastinotomy. Animals receive humane care in compliance with the Principles of Laboratory Animal Care developed by the Institute of Laboratory Animal Resources and the Guide for the Care and Use of Laboratory Animals prepared by the Institute of Laboratory Animal Resources and published by the National Institutes of Health (NIH Publication number 85-23, revised 1985). Pigs weighing 40-45 kg are anesthetized using ketamine hydrochloride (20 mg/kg intramuscular), xylazine hydrochloride (0.5 mg/kg intramuscular), and atropine sulfate (0.02 mg/kg intramuscular). Pigs are intubated and mechanically ventilated, maintaining arterial blood gas values within physiologic norms. Anesthesia is maintained with inhalation isofluorane (1.5-2%) in oxygen. An 18-gauge angiocatheter is placed in an ear vein for an intravenous infusion of 0.9% saline. Electrocardiogram leads are attached to the limbs, and the left femoral artery is instrumented with a 20-gauge angiocatheter attached to a pressure transducer to measure arterial pressure. A lidocaine bolus (3 mg/kg intravenously) is given and a lidocaine drip started at 50 ug/kg/min to suppress arrhythmias.

Animals are prepped and draped. An x-ray table allows fluoroscopy. Personnel are appropriately protected. General anesthesia is supplemented with local 1% lidocaine. The skin is incised over the third rib and the cartilage is exposed with electrocautery. The periosteum is incised longitudinally and stripped. A 5 cm segment of cartilage is cut close to the sternum. The internal thoracic artery and vein are ligated. The pleural space is entered through the rib bed. Body temperature, arterial blood gases, electrocardiogram and arterial pressure are monitored and adjusted as needed.

A 2 cm vertical pericardiotomy exposes the RV and atrial appendage. Concentric 4-0 polypropylene atrial pursestring sutures are placed and snared. The atrium is punctured through the pursestrings and a flexible guidewire is placed. Seldinger technique is used to advance an appropriately curved introducer into the RA. The introducer is rotated to align the guidemark with the expected orientation of the CS. The introducer is guided into the CS with fluoroscopy or ICE when available. The obturator is removed, and a balloon catheter is inserted for venous angiography. A multi-electrode over-the-wire lead is advanced and positioned under fluoroscopy. The introducer is stripped away and discarded. Thresholds are measured.

The purse strings around the LV lead are tied. Hemostasis is obtained. The leads are tunneled to a subcutaneous pocket and connected to a programmable pacemaker generator. The incision is closed in three layers over a red rubber catheter. The catheter is withdrawn during a valsalva maneuver. The animal is observed for pacing stability for 30 minutes and then is acutely sacrificed by injection of potassium chloride. The animals will be humanely sacrificed at the end of the experiment by arresting the heart with potassium chloride injection (1-2 mmol/kg IV) in accordance with the standards set by the American Veterinary Medical Association. The chest is opened by median sternotomy, and the heart is fixed by glutaraldehyde infusion, maintaining physiologic filling pressures as previously described. Any intrathoracic injury is noted, including pericardial or pleural effusions, cardiac chamber or CS perforations, or CS dissection. Fixed human and animal hearts will be used for in vitro testing.

In yet another aspect, the subject matter includes a family of introducers, and a single introducer manufactured to patient-specific dimensions based on 3-0 modeling from CT or MRI studies obtained within 24-72 hours of surgery.

Right parasternal mediastinotomy provides a unique opportunity to develop coronary sinus and cardiac access for research and clinical applications. This mini-thoracotomy, well tolerated by patients, can accommodate large introducers that can test custom curvatures, remote steering, and integrated intracardiac imaging. This will allow these technologies to be explored on the road to a patient-specific introducer defined by current generation CT or MRI images and three-dimensional modeling. Successful development of this product can have important impact on cardiac resynchronization, clinical electrophysiology, mitral valve repair, and mechanical circulatory assistance. A path to testing is provided by patients with advanced heart failure in whom conventional coronary sinus lead insertion cannot be implemented.

1. Protocol: This STTR will utilize 12 adult domestic pigs (40-45 kg) over one year. Animals will be of nonspecific sex. All surgery will be performed in the Institute of Comparative Medicine, P&S 20, a specialized facility equipped to conduct large animal experiments, under the supervision of a qualified animal handler. A total of 12 pigs will be utilized for the two experiments planned, six pigs assigned to each group. All animals receive humane care in compliance with the Principles of Laboratory Animal Care developed by the Institute of Laboratory Animal Resources and the Guide for the Care and Use of Laboratory Animals prepared by the Institute of Laboratory Animal Resources and published by the National Institutes of Health (NIH Publication number 85-23, revised 1985). Pigs weighing 40-45 kg are anesthetized using ketamine hydrochloride (20 mg/kg intramuscular), xylazine hydrochloride (0.5 mg/kg intramuscular), and atropine sulfate (0.02 mg/kg intramuscular). Pigs are intubated and mechanically ventilated, maintaining arterial blood gas values within physiologic norms. Anesthesia is maintained with inhalation isofluorine (1.5-2%) in 100% oxygen. An 18 gauge angiocatheter is placed in an ear vein for an intravenous infusion of 0.9% saline. Electrocardiogram leads are attached to the limbs, and the left femoral artery is instrumented with a 20 gauge angiocatheter attached to a pressure transducer to measure arterial pressure. A lidocaine bolus (3 mg/kg intravenously) is given and a lidocaine drip started at 50 μg/kg/min to suppress arrhythmias. Specific details for individual protocols are described below. Animals are prepped and draped for a right parasternal mediastinomy through the bed of the fourth rib. General anesthesia is supplemented with local 1% lidocaine in the chest wall. A transverse incision exposes the interspace, and muscles are divided with electrocautery. The periosteum is incised and elevated off of the underlying rib, which is divided and discarded. The internal thoracic artery and vein are individually ligated and the pleural space is entered. A locking retractor provides exposure. Body temperature and arterial blood gases are monitored and adjusted as needed.

Details of Aim 1: A 2 cm vertical pericardiotomy exposes the appendage of the right atrium (RA). Two concentric 4-0 polypropylene purse string sutures are placed and snared. The RA is punctured through the pursestrings, and a flexible guidewire is inserted. The Seldinger technique is used to advance an appropriately curved introducer into the RA and then into the coronary sinus (CS), with fluoroscopic guidance. A balloon catheter replaces the guidewire, and a venous angiogram is performed. An Easy-Trak over-the-wire lead is advanced and guided into position under fluoroscopy. The introducer is then stripped away and discarded. Threshold measurements are obtained. The purse string is permanently tied. The incision is closed over a red rubber catheter used to evacuate air and blood and then removed. A chest tube is not employed. The animal is awakened, maintained overnight, and then anesthetized and reimaged. The animal is humanely sacrificed. The heart is then exposed through a median sternotomy and fixed with glutaraldehyde under controlled filling pressures. Postmortem dissection is used to document any the cause of any failure of CS cannulation.

Details of Aim 2: The CS lead is secured by tying the pursestring. Screw-in, positive fixation RA and RV leads are introduced through separate pursestrings which are tied after thresholds and sensing are confirmed. The leads are brought out through the incision, tunneled to a pocket, and connected to a CRT-P generator. Closure is as described in Aim 1. Postmortem dissection is used to document any the cause of any failure of CS cannulation and any surgical complications.

Living animals are required to simulate conditions of clinical surgery. Certain design changes in instruments intended for human use cannot be accurately assessed for safety and measurement accuracy without application in living animals. Pig hearts approximate the size of the human heart and are anatomically and functionally similar. Sheep are an acceptable alternative, but protection against infection with Q-Fever is problematic. Rats and rabbits are too small to test introducers designed for humans.

Upon arrival, animals will be housed in pens and fed and cared for in the Institutional Comparative Medicine (ICM) facilities by members of our ICM staff. All animals will be housed for 3 days prior to study in the Animal Care facilities in the ICM (Black Building, 19th floor) in order to confirm a disease free state and allow the animals to become acclimated to their environment. Animals will then undergo non-survival surgery. All ICM facilities are AAALAC approved.

Animals will not be subjected to prolonged physical restraint, nutritional distress, or any other abnormal environmental conditions. The animals will be restrained only during the procedure at which time they will be under general anesthesia. Animals will be fed at all times except for an 8 hour period prior to planned general anesthesia to reduce risk of emesis and subsequent aspiration pneumonia. Animals will be anesthetized with an IM injection of xylazine (0.5 mg/kg), ketamine hydrochloride (20 mg/kg), and atropine sulfate (0.02 mg/kg). Following intubation, anesthesia will be maintained by mechanical ventilation with 1.25-2.5% isofluorane mixed with 100% oxygen. The surgeon will monitor anesthetic depth in conjunction with the staff of the ICM using the technique of corneal reflex, pain response, and muscle tone, as well as observation of respiratory patterns.

All animals will be euthanized by induction of cardiac asystole with a combination of IV potassium chloride (150 mEq) and sodium pentobarbital (100 mg/kg) under general anesthesia. Hyperkalemia causes diastolic cardiac arrest, which reduces the oxygen demand of the heart reducing the possibility of ischemic changes. This method is in accordance with the recommendations of the Panel of Euthanasia of the American Veterinary Medical Association.

### EXAMPLE 2

Methods: Anatomically derived introducers were developed in two anesthetized domestic pigs using data from computerized axial thoracic tomography. Each digitized tomogram defined a unique introducer shape, and constructed using three-dimensional modeling software and printing. Each parent pig then underwent surgery demonstrating coronary sinus lead insertion, using its custom-configured introducer. Next, with Institutional Review Board approval, 65 patients were identified who had undergone conventional endocardial coronary sinus lead insertion followed by thoracic scanning. These tomograms were used to design appropriately curved introducers for human anatomy.

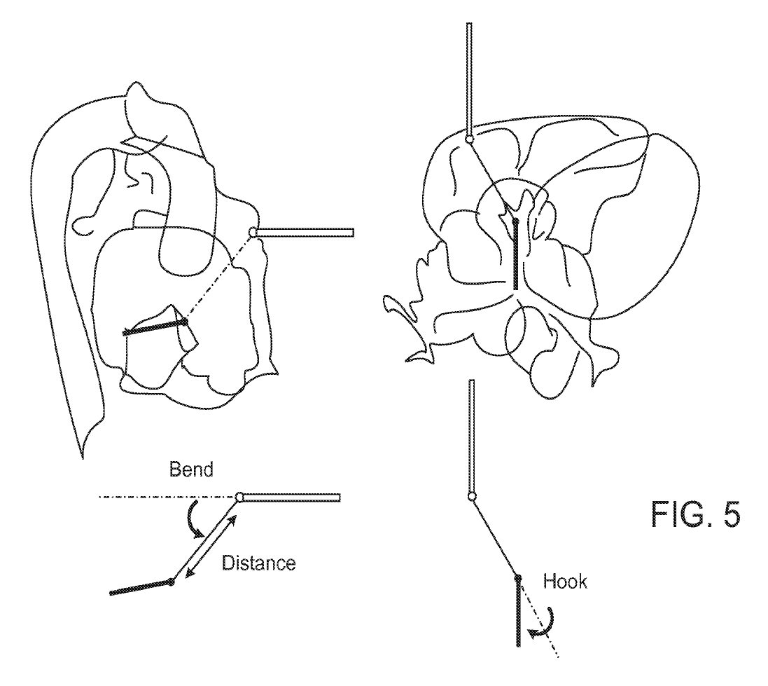

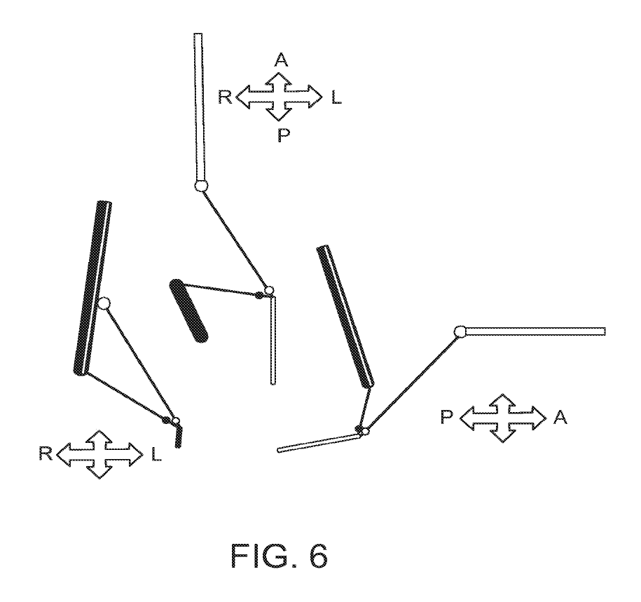

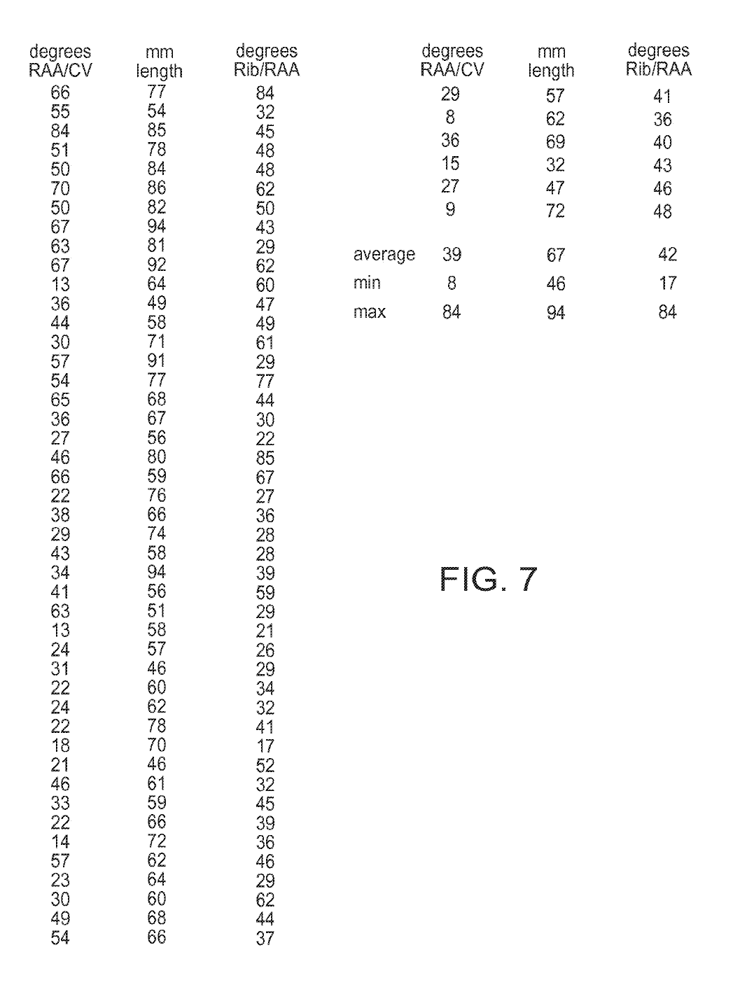

Results: Fifty-one introducer paths were defined following anatomic pathways and avoiding bends inconsistent with materials used for commercial peel-away introducers. Each path was defined by a bend and distance towards the coronary sinus ostium and a hook and twist out of plane to align with the local orientation of the coronary sinus. The average dimensions were: distance 67 mm, bend angle 47 degrees, hook angle 39 degrees and twist angle 20 degrees. FIGS. 4 and 6 show introducers having the bend, hook and twist angles, and FIG. 5 shows these angles of the introducer in situ in the heart after insertion. FIG. 7 indicate various degrees and lengths suitable for the introducer.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. An introducer configured to access a coronary sinus from an incision proximate to a right atrium appendage, the introducer comprising:
a tubular member including a first curve in a first plane corresponding to a curve of a left ventricle portion of the coronary sinus and a second curve in a different second plane corresponding to a curve of a branch vein of the coronary sinus,
wherein the first curve is disposed along a length of the tubular member and has a first angle that traverses space of an atrial appendage, a central atrium, caudad to the coronary sinus, and the second curve has an angle sufficient to align the introducer with an intrinsic curvature of the coronary sinus of a subject.

2. The introducer of claim 1, wherein the first angle includes an angle of about 40 to 50 degrees, and the second angle includes an angle of about 15 to 25 degrees.

3. The introducer of claim 1, wherein the second angle aligns the introducer with the intrinsic curvature at the origin of the coronary sinus.

4. The introducer of claim 1, wherein the introducer is configured to access the coronary sinus via right parasternal mediastinotomy.

5. The introducer of claim 1, wherein the tubular member includes an outer lumen and a central lumen.

6. The introducer of claim 5, wherein the tubular member includes a central lumen having a diameter of about 9 French, coaxially disposed within an outer lumen having a diameter of about 20 French.

7. The introducer of claim 5, wherein the tubular member includes a central lumen having a diameter of about 2 French, coaxially disposed within an outer lumen having a diameter of about 13 French.

8. The introducer of claim 5, wherein additional lumen are disposed between the outer lumen and the central lumen.

9. The introducer of claim 8, wherein a first lumen is configured to deliver a therapeutic agent to the coronary sinus, and a second lumen is configured to deliver a medical device to the coronary sinus.

10. The introducer of claim 8, wherein one lumen is configured to introduce a stylet to the coronary sinus.

11. The introducer of claim 9, wherein another lumen is configured to introduce a lead to the coronary sinus.

12. The introducer of claim 9, wherein the medical device is selected from a group consisting of: a ring, a lead, and a stent or a combination thereof.

13. The introducer of claim 1, wherein the tubular member comprises a steerable distal tip.

14. The introducer of claim 13, wherein the steerable tip is formed of a material having a different durometer than the tubular member.

15. The introducer of claim 1, wherein the tubular member is formed from SMART™ material, and further wherein the first and second curves are formed in situ.

16. An introducer configured to access a coronary sinus from an incision proximate to a right atrium appendage, the introducer comprising:
a tubular member including a first curve in a first plane corresponding to a curve of a left ventricle portion of the coronary sinus and a second curve in a different second plane corresponding to a curve of a branch vein of the coronary sinus, wherein the tubular member includes a central lumen having a diameter of about 9 French, coaxially disposed within an outer lumen having a diameter of about 20 French.

17. The introducer of claim 16, wherein the first curve is disposed along a length of the tubular member and has a first angle that traverses space of an atrial appendage, a central atrium, caudad to the coronary sinus, and the second curve has an angle sufficient to align the introducer with an intrinsic curvature of the coronary sinus of a subject.

18. The introducer of claim 17, wherein the first angle includes an angle of about 40 to 50 degrees, and the second angle includes an angle of about 15 to 25 degrees.

19. The introducer of claim 17, wherein the second angle aligns the introducer with the intrinsic curvature at the origin of the coronary sinus.

20. The introducer of claim 16, wherein the introducer is configured to access the coronary sinus via right parasternal mediastinotomy.

21. The introducer of claim 16, wherein the tubular member includes an outer lumen.

22. The introducer of claim 21, wherein additional lumen are disposed between the outer lumen and the central lumen.

23. The introducer of claim 22, wherein a first lumen is configured to deliver a therapeutic agent to the coronary sinus, and a second lumen is configured to deliver a medical device to the coronary sinus.

24. The introducer of claim 22, wherein one lumen is configured to introduce a stylet to the coronary sinus.

25. The introducer of claim 23, wherein another lumen is configured to introduce a lead to the coronary sinus.

26. The introducer of claim 23, wherein the medical device is selected from a group consisting of: a ring, a lead, and a stent or a combination thereof.

27. The introducer of claim 16, wherein the tubular member comprises a steerable distal tip.

28. The introducer of claim 27, wherein the steerable tip is formed of a material having a different durometer than the tubular member.

29. The introducer of claim 16, wherein the tubular member is formed from SMART™ material, and further wherein the first and second curves are formed in situ.

30. An introducer configured to access a coronary sinus from an incision proximate to a right atrium appendage, the introducer comprising:
an outer tubular member including a first curve in a first plane corresponding to a curve of a left ventricle portion of the coronary sinus and a second curve in a different second plane corresponding to a curve of a branch vein of the coronary sinus, and
an inner tubular member coaxially disposed within the outer tubular member, wherein at least one lumen is disposed between the inner and outer tubular members.

31. The introducer of claim 30, wherein the inner tubular member includes a first curve in a first plane and a second curve in a different second plane.

32. The introducer of claim 30, wherein the first curve is disposed along a length of the outer tubular member and has a first angle that traverses space of an atrial appendage, a central atrium, caudad to the coronary sinus, and the second curve has an angle sufficient to align the introducer with an intrinsic curvature of the coronary sinus of a subject.

33. The introducer of claim 31, wherein the first angle includes an angle of about 40 to 50 degrees, and the second angle includes an angle of about 15 to 25 degrees.
